(19) United States
(12) Patent Application Publication
(10) Pub. No.: US 2002/0147366 A1
(43) Pub. Date: Oct. 10, 2002

(54) Alkoxylated tertiary and quaternary amine surfactants

(76) Inventors: Michael S. Wiggins, Lansdale, PA (US); Reuben Grinstein, Tucson, AZ (US); Dharmesh Chovatia, Bensalem, PA (US)

Correspondence Address: COGNIS CORPORATION, 2500 RENAISSANCE BLVD., SUITE 200, GULPH MILLS, PA, 19406

(21) Appl. No.: 09/896,313

(22) Filed: Jun. 29, 2001

Related U.S. Application Data
(60) Provisional application No. 60/216,749, filed on Jul. 7, 2000.

(51) Int. Cl. C 07C 2 9/20

(52) U.S. Cl. 564/281; 564/402; 564/480

(57) ABSTRACT

Process for the preparation of alkoxylated tertiary and quaternary amine surfactants by reductive amination of alkoxylated alcohols with anhydrous ammonia or primary or secondary amines or polyamines, optionally followed by quaternization of the alkoxylated tertiary amines with a quaternizing agent such as an aliphatic halide.

The invention also relates to the above surfactant products and methods for their use.

### CROSS-REFERENCE TO RELATED APPLICATIONS

This application claims the benefit of copending provisional application serial No. 60/216,749, filed on Jul. 7, 2000, the entire contents of which are incorporated herein by reference.

### BACKGROUND OF THE INVENTION

The development of new surfactants which will have a number of commercial uses, both specialized uses and multiple uses, is an ongoing research project carried out by many commercial enterprises throughout the world.

There is, for example, a continuing need for surfactants having good water solubility and low foam characteristics.

### BRIEF SUMMARY OF THE INVENTION

There has now been discovered a process for preparing alkoxylated tertiary and quaternary amine surfactants having excellent water solubility combined with low foam properties.

The alkoxylated tertiary amines are prepared by the reductive amination of alkoxylated alcohols with anhydrous ammonia or primary or secondary amines or diamines or polyamines.

The alkoxylated quaternary amine surfactants of the invention are prepared by reacting the above alkoxylated tertiary amines with a quaternizing agent. The alkoxylated quaternary amine surfactants have an even greater water solubility than the alkoxylated tertiary amines of the invention.

These surfactant products, many of which are highly branched, have a number of uses including utility as defoamers, as high mobility low foam surfactants, as viscosity reducers and crystallization inhibitors for high foaming alkyl polyglycoside surfactants and other high foaming surfactants, and as surfactants for emulsion polymerizations and as protective colloids for use therein.

### DETAILED DESCRIPTION OF THE INVENTION

Other than in the operating examples, or where otherwise indicated, all numbers expressing quantities of ingredients or reaction conditions used herein are to be understood as modified in all instances by the term “about”.

The present process is carried out by reacting an aliphatic alkoxylated alcohol or an aromatic alboxylated alcohol, or a mixture of such alcohols, preferably those of formula I below:

R¹(OA)ₙ—OH (I)

wherein R¹ can be an aliphatic group containing from 4 to 36 carbon atoms, preferably from 8 to 22 carbon atoms, or an aromatic group, e.g. phenyl, naphthyl, anthryl, and the like, wherein the aromatic group can be alkyl substituted, e.g. by a C₁-C₁₈ alkyl group, n is a number of from 1 to 200, and each OA group is independently an ethyleneoxy, 1,2-propyleneoxy, or 1,2-butyleneoxy group, with anhydrous ammonia or an amine which is optionally alkoxylated. The amine can be primary or secondary monoamines, or diamines or polyamines. As stated above, the foregoing amines can also be alkoxylated, e.g. can contain from 1 to 300 ethyleneoxy, 1,2-propyleneoxy, or 1,2-butyleneoxy groups, or a mixture thereof in the molecule. The amine is preferably a compound of formula II, III or IV below:

R²R³NH (II)

wherein R² can be a C₁-C₂₂ linear or branched alkyl group, a C₅-C₁₀ cycloalkyl group, or a linear or branched alkyl group in which a C₅-C₁₀ cycloalkyl group is present provided the total number of carbon atoms is from 6 to 22, and R³ is hydrogen or independently from the R² group is a C₁-C₂₂ alkyl group; or R² and R³ together with the nitrogen atom can be a heterocyclic amine, e.g. piperazine, piperadine, and any of the foregoing with one or two double bonds;

(H₂N)ₐ(R⁴NH)ₓR⁴NH₂ (III)

wherein R⁴ is a C₂₋₂₀ linear or branched aliphatic group, a C₅-C₁₀ cycloaliphatic group, or a linear or branched aliphatic group in which a C₅-C₁₀ cycloaliphatic group may also optionally be present provided the total number of carbon atoms is from 6 to 22, a is 0 or 1, and x is a number of from 0 to 10, provided however that when x is 0, a is 1;

H₂N—CH(R⁵)CH₂—O—R⁶—CH₂CH(R⁵)—NH₂ (IV)

wherein R⁶ represents a polyoxyalkylene chain having the structural formula:

(O—CH₂—CH₂—)ₐ(O—CH₂—CH(R⁷))_{b}

wherein R⁷ is a monovalent organic radical selected from the group consisting of C₁ to C₄ aliphatic hydrocarbons, ‘a’ designates a number of ethyleneoxy groups (O—CH₂—CH₂), ‘b’ designates the number of monosubstituted ethyleneoxy groups (O—CH₂—CH(R⁷)), the sum of ‘a’ and ‘b’ is equal to or greater than 10 but less than or equal to 300, provided that for any values of a and b the sequence of ethyleneoxy and monosubstituted ethyleneoxy groups within a polyoxyalkylene chain may be completely random and/or there may be blocks of ethyleneoxy and/or monosubstituted ethyleneoxy groups, and R⁵ designates hydrogen or a monovalent organic radical selected from the group consisting of C₁ to C₄ aliphatic hydrocarbon groups, in the presence of an effective quantity of a reduction catalyst, e.g. a nickel catalyst such as nickel on a support reduced with hydrogen, other transition metals such as cobalt, noble metal reduction catalysts such as palladium, platinum, and the like.

The reaction product is then separated from the reduction catalyst, e.g. by filtration. If the product is a solid or is highly viscous, the reaction product can be dissolved in a suitable solvent such as water, a lower alcohol, a water-alcohol mixture, a ketone, an aromatic solvent, etc.

The reaction product can then be further purified, if desired, by distilling off the solvent if present and removing unreacted starting materials and volatile by-products under vacuum.

The above process can be carried out at a temperature in the range of 100 to 300° C., preferably 100 to 200° C., and under pressures ranging from autogenous to 200 psig. The equivalent ratio of ammonia or amine, based on available hydrogen atoms on the nitrogen atoms in the ammonia or amine, to alkoxylated alcohol can range from 1:1 to 1:10, preferably from 1:1 to 1:3.

In the aliphatic alkoxylated alcohol of formula I, the R¹ group is preferably a straight or branched chain alkyl group containing from 8 to 20 carbon atoms, more preferably from 16 to 20 carbon atoms. However, the R¹ group can also be an aromatic group, a linear or branched alkenyl or alkynyl group, a saturated carbocyclic moiety, an unsaturated carbocyclic moiety having one or more multiple bonds, a saturated heterocyclic moiety, or an unsaturated heterocyclic moiety having one or more multiple bonds, including aromatic heterocyclic moieties. The number of alkoxy groups, i.e. the value for n in formula I, is preferably from 1 to 10, and more preferably from 2 to 8. The OA groups are preferably all ethyleneoxy groups.

In the amine compounds of formula II, the R² group is preferably a C₁-C₆ alkyl group, and R³ is hydrogen or independently a C₁-C₆ alkyl group.

The amines of formula III can be diamines or polyamines. The diamines can be one or more aliphatic or cycloaliphatic or cycloaliphatic-containing diamines having from 2 to 20 carbon atoms. Preferred are the alkylene diamines, such as ethylene diamine, 1,3-diaminopropane, 1,4-diaminobutane, 1,6-hexamethylene diamine, bis-(4-cyclohexylamine)methane, 2,2-bis-(4-cyclohexylamine)propane, polyglycol diamines, isophorone diamine, cyclohexanebis(methylamines), dimer diamine, polyether diamines, methyl pentamethylene diamine, and piperazine. These diamine compounds are all prepared by well known methods and many are commercially available. Particularly preferred are the straight chain alkylene diamines of 2 to 20 carbon atoms, especially ethylene diamine and hexamethylene diamine, and cycloalkylene diamines, especially 4,4′-methylenebis cyclohexylamine and piperazine.

The polyamines of formula III, i.e. compounds of formula III wherein x is 1 to 10 are preferably compounds in which the R⁴ group is a C₂-C₆ alkylene group and x is a number of from 1 to 6. Also preferred are polyethyleneimines.

The alkoxylated quaternary amine surfactants of the invention are prepared by reacting the tertiary amine reaction product of the above process with a quaternizing agent. Quaternizing agents that can be used include compounds of formula V below:

R⁸X (V)

in which R⁸ is a C₁-C₁₀ alkyl group or a phenyl-C₁-C₁₀ alkyl group, and x is chlorine, bromine or iodine, preferably chlorine or bromine. The R⁸ group is preferably a C₁-C₈ alkyl group or the benzyl group. Other quaternizing agents are alkylene and arylene oxides, e.g. ethylene oxide, propylene oxide, butylene oxide, phenylene oxide, and the like.

The reaction of the tertiary amine reaction products and the quaternizing agents can be carried out at a temperature in the range of 20° C. to 100° C., preferably from 20 to 40° C.

The products of the reaction between the alkoxylated alcohol of formula I and the amine of formula II have the formula VII below:

R¹(OA)ₙNR²R³ (VII)

wherein R¹ is an aliphatic group containing from 4 to 36 carbon atoms, or an optionally alkyl substituted aromatic group; n is a number of from 1 to 200, and each OA group is independently an ethyleneoxy, 1,2-propyleneoxy, or 1,2-butyleneoxy group; R² is a C₁-C₂₂ linear or branched alkyl group, a C₅-C₁₀ cycloalkyl group, or a linear or branched alkyl group in which a C₅-C₁₀ cycloalkyl group is present provided the total number of carbon atoms is from 6 to 22, and R³ is hydrogen or independently from the R² group is a C₁-C₂₂ alkyl group; or R² and R³ together with the nitrogen atom is a heterocyclic amine.

The products of the reaction between the alkoxylated alcohol of formula I and the amine of formula III have the formula VIII below:

(R¹(OA)ₙNH)ₐ(R⁴NH)ₓR⁴NH(AO)ₙR¹ (VIII)

wherein R¹ is an aliphatic group containing from 4 to 36 carbon atoms, or an optionally alkyl substituted aromatic group; n is a number of from 1 to 200, and each OA group is independently an ethyleneoxy, 1,2-propyleneoxy, or 1,2-butyleneoxy group; R⁴ is a C₂₋₂₀ linear or branched aliphatic group, a C₅-C₁₀ cycloaliphatic group, or a linear or branched aliphatic group in which a C₅-C₁₀ cycloaliphatic group may also optionally be present provided the total number of carbon atoms is from 6 to 22, a is 0 or 1, and x is a number of from 0 to 10, provided however that when x is 0, a is 1.

The products of the reaction between the alkoxylated alcohol of formula I and the amine of formula IV have the formula IX below:

R¹(OA)ₙNH—CH(R⁵)CH₂—O—R⁶—CH₂CH(R⁵)—NH(AO)ₙR¹ IX

wherein R¹ is an aliphatic group containing from 4 to 36 carbon atoms, or an optionally alkyl substituted aromatic group; n is a number of from 1 to 200, and each OA group is independently an ethyleneoxy, 1,2-propyleneoxy, or 1,2-butyleneoxy group; R⁶ represents a polyoxyalkylene chain having the structural formula:

(O—CH₂—CH₂—)ₐ(O—CH₂—CH(R⁷))_{b}

wherein R⁷ is a monovalent organic radical selected from the group consisting of C₁ to C₄ aliphatic hydrocarbons; ‘a’ designates a number of ethyleneoxy groups (O—CH₂—CH₂); ‘b’ designates the number of monosubstituted ethyleneoxy groups (O—CH₂—CH(R⁷)); the sum of ‘a’ and ‘b’ is equal to or greater than 10 but less than or equal to 300, provided that for any values of a and b the sequence of ethyleneoxy and monosubstituted ethyleneoxy groups within a polyoxyalkylene chain can be completely random and/or there can be blocks of ethyleneoxy and/or monosubstituted ethyleneoxy groups; and R⁵ designates hydrogen or a monovalent organic radical selected from the group consisting of C₁ to C₄ aliphatic hydrocarbon groups.

The alkoxylated tertiary and amine surfactants produced by the process of the invention can be used as phase transfer catalysts and metal ion chelation agents to carry the metal ions from an aqueous phase into a water-immiscible organic solvent phase. This utility is important in the refining of metals from metal ores.

They can also be used in from 0.1 to 20% by weight, based on solids content, as a surfactant and protective colloid in hydrophilic emulsion polymer latexes, e.g. vinyl acrylic latexes, to produce a stable latex.

The addition, they can be used as a surfactant component of cleaning, and laundry and dishwasher detergent compositions where good water solubility, high mobility, and low foaming properties in aqueous solutions are desired for the surfactant component thereof. For this use, from 1 to 50% by weight, based on solids content, can be present in the compositions.

They can also be used to control foaming in otherwise high foaming cleaning compositions.

Aqueous cleaning compositions exhibit a tendency toward foaming because they contain surface active agents such as soaps, and synthetic detergents. In many instances, such cleaning compositions produce excessive foam and the user must use substances known as anti-foaming agents or defoamers. Some defoamers such as silicones tend to interfere with the function of the cleaning compositions in that unwanted residues are left after the cleaners are wiped off while others are environmentally unacceptable because they are not biodegradable.

Alkyl polyglycosides are a class of nonionic surfactants that exhibit significantly higher foaming profiles than other nonionic surfactants, such as alcohol ethyleneoxylates. In fact, it can be said that the foaming tendencies of alkyl polyglycosides more closely resemble those of anionic surfactants, such as alcohol sulfates, than the foaming tendencies of other nonionic surfactants. This higher foaming tendency makes the use of alkyl polyglycosides undesirable for many applications, e.g. cleaning-in-place for food processing plants, high pressure spray cleaning, bottle washing, floor cleaners and automatic dishwashing, wherein high levels of foam interfere with the cleaning and rinsing operation and reduce the efficiency of the operation.

Low foam nonionics, such as EO/PO block copolymers, can be used to reduce the foaming properties of alkyl polyglycoside surfactants, but these materials have undesirable properties, e.g. low biodegradability, relatively high aquatic toxicity and poor caustic compatibility.

The alkoxylated tertiary and quaternary amine surfactants of the invention act as anti-foam agents in compositions, e.g. cleaning compositions, containing alkyl polyglycoside surfactants or other high foaming surfactants. They are added to the cleaning compositions in an amount sufficient to reduce or eliminate foam when used in aqueous solution. The amount required to eliminate and/or decrease foam is defined as a defoaming effective amount and will vary from one instance to another depending upon the nature of the surfactant or mixture of surfactants and the defoaming effect desired. A defoaming effective amount will be readily determinable by one of ordinary skill in the art. When the surfactant is one or more alkyl polyglycoside, the defoaming effective amount will typically vary from a weight ratio of alkyl polyglycoside/defoamer of 4.0/1.0 to about 1.0/1.0.

The alkyl polyglycosides are well known to the art and can be represented by formula VI below:

R⁹O(R¹⁰O)_{b}(Z)ₐ (VI)

wherein R⁹ is a monovalent organic radical having from 6 to 30 carbon atoms; R¹⁰ is a divalent alkylene radical having from 2 to 4 carbon atoms; Z is a saccharide residue having 5 or 6 carbon atoms; b is a number having a value from 0 to 12; and a is a number having a value from 1 to 6. Preferred alkyl polyglycosides used in cleaning compositions have the formula VI wherein Z is a glucose residue and b is zero. Such alkyl polyglycosides are commercially available, for example, as APG®, GLUCOPON®, or PLANTAREN® surfactants from Cognis Corporation, Ambler, Pa. 19002. Examples of such surfactants include but are not limited to:

1. APG® 225 Surfactant—an alkyl polyglycoside in which the alkyl group contains 8 to 10 carbon atoms and having an average degree of polymerization of 1.7.

2. APG® 425 Surfactant—an alkyl polyglycoside in which the alkyl group contains 8 to 16 carbon atoms and having an average degree of polymerization of 1.5.

3. APG® 625 Surfactant—an alkyl polyglycoside in which the alkyl group contains 12 to 16 carbon atoms and having an average degree of polymerization of 1.6.

4. APG® 325 Surfactant—an alkyl polyglycoside in which the alkyl group contains 9 to 11 carbon atoms and having an average degree of polymerization of 1.5.

5. GLUCOPON® 600 Surfactant—an alkyl polyglycoside in which the alkyl group contains 12 to 16 carbon atoms and having an average degree of polymerization of 1.4.

6. PLANTAREN® 2000 Surfactant—a C8-16 alkyl polyglycoside in which the alkyl group contains 8 to 16 carbon atoms and having an average degree of polymerization of 1.5.

7. PLANTAREN® 1300 Surfactant—a C12-16 alkyl polyglycoside in which the alkyl group contains 12 to 16 carbon atoms and having an average degree of polymerization of 1.6.

8. GLUCOPON® 220 Surfactant—an alkyl polyglycoside in which the alkyl group contains 8 to 10 carbon atoms and having an average degree of polymerization of 1.5.

The invention will be illustrated, but not limited, by the following examples.

### EXAMPLES

### Example 1

Into a stirrer-equipped autoclave reactor with condenser and decant system there are added 1000 gms (3 moles) of isodecyl alcohol with an average 4 moles of ethylene oxide (TRYCOL®) and 15 gms (1.5 wt. %) Girdler G49B nickel catalyst. Stirring is started. A vacuum of 20″ Hg is applied, and the vacuum is released with nitrogen gas. The latter procedure is repeated twice. The reactor is then closed and pressurized to 60 psig with hydrogen. The reactor is heated to 200° C. while maintaining 60 psig pressure. Then 17 grams (1.0 mole) of anhydrous ammonia is added. After 1 hour the hydrogen pressure is reduced to 30 psig and held for 1 hour. A sample from the reactor is removed and titrated for total amine and tertiary amine values. The hydrogen pressure is released, a vacuum of 20″ Hg is applied, and the vacuum is released with nitrogen. The vacuum and nitrogen release is repeated several times. The liquid product is then filtered to remove the catalyst. The resulting product is tri (isodecanol.4EO) amine.

### Example 2

Into a stirrer-equipped autoclave reactor with condenser and decant system there are added 1800 gms (5 moles) of dodecyl alcohol with an average 4 moles of ethylene oxide (TRYCOL® 5882), 103 gms (1.0 mole) of diethylenetriamine, and 30 gms (1.5 wt %) Girdler G49B nickel catalyst. Stirring is started. A vacuum of 20″ Hg is applied, and the vacuum is released with nitrogen gas. The latter procedure is repeated twice. The reactor is then closed and pressurized to 60 psig with hydrogen. The reactor is heated to 200° C. while maintaining 60 psig pressure. After 1 hour the hydrogen pressure is reduced to 30 psig and held for 1 hour. A sample from the reactor is removed and titrated for total amine and tertiary amine values. The hydrogen pressure is released, a vacuum of 20″ Hg is applied, and the vacuum is released with nitrogen. The vacuum and nitrogen release is repeated several times. The liquid product is then filtered to remove the catalyst. The resulting product is penta (dodecanol.4EO) diethylenetriamine.

### Example 3

The process of Example 2 is repeated except that the reactants are 1100 grams (4.2 moles) of dodecyl alcohol.4EO (TRYCOL®5952) and 180 grams (0.1 moles) of polyethyleneimine, with 60 grams (5 wt %) Girdler G49B nickel catalyst.

The resulting product is poly(isodecanol.4EO) amine.

### Example 4

The process of Example 1 is repeated except that the reactants are 667 grams (2 moles) of isodecyl alcohol.4EO (TRYCOL®5950), 100 grams (1OH equivalent) of PEG-200, with 15 grams (1.5 wt %) Girdler G49B nickel catalyst. The quantity of anhydrous ammonia used in the process is also 17 grams (1.0 mole). The product is tetra(isodecanol.4EO) PEG diamine.

### Example 5

The product from Example 4 is placed in the autoclave reactor. The reactor is closed off and pressurized to 30 psig with methyl chloride. The reactor is heated to 80° C. and stirred for 1 hour or until the pressure stops dropping. The pressure is released, 20″Hg vacuum is applied, and the vacuum released with nitrogen gas. The vacuum application and nitrogen release is repeated several times.

The resulting product is the quaternary ammonium salt of tetra (isodecanol•4EO) PEG diamine.

What is claimed is:

1. A process for the preparation of alkoxylated amines comprising the steps of
A) reacting (a) at least one aliphatic alkoxylated alcohol with (b) a nitrogen-containing compound selected from the group consisting of (i) anhydrous ammonia, (ii) a primary or secondary amine which is optionally alkoxylated, and (iii) a diamine or polyamine which is optionally alkoxylated, in the presence of a reduction catalyst to form an alkoxylated tertiary amine reaction product;
B) separating the alkoxylated tertiary amine reaction product from the reduction catalyst; and optionally,
C) reacting the alkoxylated tertiary amine reaction product with a quaternizing agent to form an alkoxylated quaternary ammonium compound.

2. The process of claim 1 wherein the reduction catalyst in step A) is a nickel catalyst.

3. The process of claim 1 wherein step B) is carried out by dissolving the alkoxylated tertiary amine reaction product in a solvent and filtering off the reduction catalyst.

4. The process of claim 3 wherein after step B) and before optional step C) the alkoxylated tertiary amine reaction product is purified by distilling off the solvent and removing unreacted starting materials and volatile by-products under vacuum.

5. The process of claim 1 wherein step A) is carried out at a temperature in the range of from about 100 to about 300° C.

6. The process of claim 5 wherein said temperature is from about 100 to about 200° C.

7. The process of claim 1 wherein the equivalent ratio of ammonia or amine to alkoxylated alcohol is from about 1:1 to about 1:10, based on available hydrogen atoms on the nitrogen atoms in the ammonia or amine.

8. The process of claim 1 wherein step C) is carried out at a temperature in the range of from about 20° C. to about 100° C.

9. An alkoxylated tertiary amine formed by steps A) and B) of the process of claim 1.

10. A quaternary alkoxylated amine formed by the process of steps A), B) and C) of claim 1.

11. A process for the preparation of alkoxylated amines comprising steps of
A) reacting (a) at least one aliphatic alkoxylated alcohol or alkoxylated aromatic alcohol of the formula
R¹(OA)ₙ—OH (I)
wherein R¹ is a hydrocarbon group containing from 4 to 36 carbon atoms, n is a number of from 1 to 200, and each OA group is independently an ethyleneoxy, 1,2-propyleneoxy, 1,2-butyleneoxy or phenylethyleneoxy group; with (b) a nitrogen-containing compound selected from the group consisting of
i) anhydrous ammonia,
ii) a primary or secondary amine of the formula
R²R³NH (II)
wherein R² is a C₁-C₂₂ linear or branched alkyl group, a C₅-C₁₀ cycloalkyl group, or a linear or branched alkyl group in which a C₅-C₁₀ cycloalkyl group aromatic is present provided the total number of carbon atoms is from 6 to 22, and R³ is hydrogen or independently from the R² group is a C₁-C₂₂ alkyl group, or R² and R³ together with the nitrogen atom can be a hetercyclic amine;
iii) a diamine or polyamine of the formula
(H₂N)ₐ(R⁴NH)ₓR⁴NH₂ (III)
wherein R⁴ is a C₂₋₂₀ linear or branched aliphatic group, a C₅-C₁₀ cycloaliphatic group or a linear or branched aliphatic group in which a C₅-C₁₀ cycloaliphatic group may also optionally be present provided the total number of carbon atoms is from 6 to 22, a is 0 or 1, and x is a number of from 0 to 10, provided however that when x is 0, a is 1, and
iv) an alkoxylated amine of the formula
H₂N—CH(R⁵)CH₂—O—R⁶—CH₂CH(R⁵)—NH₂ (III)
wherein R⁶ represents a polyoxyalkylene chain having the structural formula:
(O—CH₂—CH₂—)ₐ(O—CH₂—CH(R⁷))_{b} (IV)
wherein R⁷ is a monovalent organic radical selected from the group consisting of C₁ to C₄ aliphatic hydrocarbons, ‘a’ designates a number of ethyleneoxy groups (O—CH₂—CH₂), ‘b’ designates the number of monosubstituted ethyleneoxy groups (O—CH₂—CH(R⁷)), the sum of ‘a’ and ‘b’ is equal to or greater than 10 but less than or equal to 300, provided that for any values of a and b the sequence of ethyleneoxy and monosubstituted ethyleneoxy groups within a polyoxyalkylene chain may be completely random and/or there may be blocks of ethyleneoxy and/or monosubstituted ethyleneoxy groups, and R⁵ designates hydrogen or a monovalent organic radical selected from the group consisting of C₁ to C₄ aliphatic hydrocarbons, in the presence of a reduction catalyst to form an alkoxylated tertiary amine reaction product;
B) separating the alkoxylated tertiary amine reaction product from the reduction catalyst; and optionally,
C) reacting the alkoxylated tertiary amine reaction product with a quaternizing agent to form an alkoxylated quaternary ammonium compound.

12. The process of claim 11 wherein step C) is carried out with a quaternizing agent of formula V
R⁸X (V)
in which R⁸ us a C₁-C₁₀ alkyl group or a phenyl-C₁-C₁₀ alkyl group, and x is chlorine, bromine, or iodine.

13. The process of claim 11 wherein the reduction catalyst in step A) is a nickel catalyst.

14. The process of claim 11 wherein step B) is carried out by dissolving the alkoxylated tertiary amine reaction product in a solvent and filtering off the reduction catalyst.

15. The process of claim 14 wherein after step B) and before optional step C) the alkoxylated tertiary amine reaction product is purified by distilling off the solvent and removing unreacted starting materials and volatile by-products under vacuum.

16. The process of claim 11 wherein step A) is carried out at a temperature in the range of from about 100 to about 300° C.

17. The process of claim 16 wherein said temperature is from about 100 to about 200° C.

18. The process of claim 11 wherein the equivalent ratio of ammonia or amine to alkoxylated alcohol is from about 1:1 to about 1:10.

19. The process of claim 11 wherein step C) is carried out at a temperature in the range of from about 20° C. to about 100° C.

20. An alkoxylated tertiary amine formed by steps A) and B) of the process of claim 11.

21. A quaternary alkoxylated amine formed by steps A), B) and C) of the process of claim 11.

22. In a process for refining metals from metal ores in which the metal ions are present in an aqueous phase in contact with a water-immiscible organic solvent phase, the improvement wherein an alkoxylated amine of claim 26 is used to carry the metal ions from the aqueous phase into the organic solvent phase.

23. In a process for the preparation of hydrophilic emulsion polymer latexes, the improvement wherein a surfactant-effective quantity of an alkoxylated amine of claim 26 is present therein.

24. In a cleaning composition, a laundry detergent composition, or a dishwasher detergent composition, the improvement wherein a surfactant-effective quantity of an alkoxylated amine of claim 26 is present therein.

25. In a composition containing a surfactant which produces an undesirably high level of foam in water, the improvement wherein the composition contains an anti-foaming effective quantity of an alkoxylated amine of claim 26.

26. The reductive ammonation product of an alkoxylated aliphatic or aromatic alcohol and ammonia or an optionally alkoxylated amine.

27. The product of claim 26 wherein the alkoxylated alcohol has the formula:
R¹(OA)ₙ—OH (I)
wherein R¹ can be an aliphatic group containing from 4 to 36 carbon atoms, or an optionally alkyl substituted aromatic group; n is a number of from 1 to 200; and each OA group is independently an ethyleneoxy, 1,2-propyleneoxy, or 1,2-butyleneoxy group.

28. The reductive ammonation product of claim 26 which is quaternized with a compound of the formula R⁸x or an alkylene oxide or an arylene oxide, wherein R⁸ is a C₁-C₁₀ alkyl group or a phenyl-C₁-C₁₀ alkyl group, and x is chlorine, bromine, or iodine.

29. The product of claim 26 which has the formula VII below:
R¹(OA)ₙNR²R³ (VI)
wherein R¹ is an aliphatic group containing from 4 to 36 carbon atoms, or an optionally alkyl substituted aromatic group; n is a number of from 1 to 200, and each OA group is independently an ethyleneoxy, 1,2-propyleneoxy, or 1,2-butyleneoxy group; R² is a C₁-C₂₂ linear or branched alkyl group, a C₅-C₁₀ cycloalkyl group, or a linear or branched alkyl group in which a C₅-C₁₀ cycloalkyl group is present provided the total number of carbon atoms is from 6 to 22, and R³ is hydrogen or independently from the R² group is a C₁-C₂₂ alkyl group; or R² and R³ together with the nitrogen atom is a heterocyclic amine.

30. The product of claim 29 wherein the product is quaternized with a compound of the formula R⁸x or an alkylene oxide or an arylene oxide, wherein R⁸ is a C₁-C₁₀ alkyl group or a phenyl-C₁-C₁₀ alkyl group, and x is chlorine, bromine, or iodine.

31. The product of claim 26 which has the formula VIII below:
(R¹(OA)ₙNH)ₐ(R⁴NH)ₓR⁴NH(OA)ₙR¹ (VIII)
wherein R¹ is an aliphatic group containing from 4 to 36 carbon atoms, or an optionally alkyl substituted aromatic group; n is a number of from 1 to 200; and each OA group is independently an ethyleneoxy, 1,2-propyleneoxy, or 1,2-butyleneoxy group; R⁴ is a C₂₋₂₀ linear or branched aliphatic group, a C₅-C₁₀ cycloaliphatic group, or a linear or branched aliphatic group in which a C₅-C₁₀ cycloaliphatic group may also optionally be present provided the total number of carbon atoms is from 6 to 22, a is 0 or 1, and x is a number of from 0 to 10, provided however that when x is 0, a is 1.

32. The reductive ammonation product of claim 31 which is quaternized with a compound of the formula R⁸x or an alkylene oxide or an arylene oxide, wherein R⁸ is a C₁-C₁₀ alkyl group or a phenyl-C₁-C₁₀ alkyl group, and x is chlorine, bromine, or iodine.

33. The product of claim 26 which has the formula IX below:
R¹(OA)ₙNH—CH(R⁵)CH₂—O—R⁶—CH₂CH(R⁵)—NH(OA)ₙR¹ (IX)
wherein R¹ is an aliphatic group containing from 4 to 36 carbon atoms, or an optionally alkyl substituted aromatic group; n is a number of from 1 to 200, and each OA group is independently an ethyleneoxy, 1,2-propyleneoxy, or 1,2-butyleneoxy group; R⁶ represents a polyoxyalkylene chain having the structural formula:
(O—CH₂—CH₂—)ₐ(O—CH₂—CH(R⁷))_{b}
wherein R⁷ is a monovalent organic radical selected from the group consisting of C₁ to C₄ aliphatic hydrocarbons; ‘a’ designates a number of ethyleneoxy groups (O—CH₂—CH₂); ‘b’ designates the number of monosubstituted ethyleneoxy groups (O—CH₂—CH(R⁷)); the sum of ‘a’ and ‘b’ is equal to or greater than 10 but less than or equal to 300, provided that for any values of a and b the sequence of ethyleneoxy and monosubstituted ethyleneoxy groups within a polyoxyalkylene chain can be completely random and/or there can be blocks of ethyleneoxy and/or monosubstituted ethyleneoxy groups; and R⁵ designates hydrogen or a monovalent organic radical selected from the group consisting of C₁ to C₄ aliphatic hydrocarbon groups.

34. The reductive ammonation product of claim 33 which is quaternized with a compound of the formula R⁸x or an alkylene oxide or an arylene oxide, wherein R⁸ is a C₁-C₁₀ alkyl group or a phenyl-C₁-C₁₀ alkyl group, and x is chlorine, bromine, or iodine.
